(19) United States
(12) Patent Application Publication
(10) Pub. No.: US 2013/0095493 A1
(43) Pub. Date: Apr. 18, 2013

(54) IDENTIFICATION OF THE GENE NOTCH3 AS A NOVEL BIOMARKER FOR HUMAN METASTATIC MELANOMA

(71) Applicants: Rhoda Myra Alani, Baltimore, MD (US); Jason David Howard, Catonsville, MD (US)

(72) Inventors: Rhoda Myra Alani, Baltimore, MD (US); Jason David Howard, Catonsville, MD (US)

(73) Assignee: THE JOHNS HOPKINS UNIVERSITY, Baltimore, MD (US)

(21) Appl. No.: 13/644,131

(22) Filed: Oct. 3, 2012

Related U.S. Application Data
(60) Provisional application No. 61/542,407, filed on Oct. 3, 2011.

(51) Int. Cl. G01N 33/68; C12Q 1/02

(52) U.S. Cl. CPC G01N 33/6893; C12Q 1/025
USPC 435/6.12; 435/7.23; 435/7.92; 435/6.13

(57) ABSTRACT

The present invention provides evidence that Notch3 gene upregulation is associated with invasive metastatic melanoma. Assays for quantifying Notch3 expression in biological samples and methods of use for diagnosis, and assaying progression and treatment outcome using these assays are also provided. Methods of screening for compounds which inhibit Notch3 expression are also provided.

### REFERENCE TO RELATED APPLICATIONS

This application claims the benefit of U.S. Provisional Patent Application No. 61/542,407, filed on Oct. 3, 2011, which is hereby incorporated by reference for all purposes as if fully set forth herein.

### BACKGROUND OF THE INVENTION

The incidence of melanoma is increasing at the highest rate for any form of cancer in the United States and the current lifetime risk in the US is 1 in 68. Presently, there are few effective systemic therapies to treat advanced stages of melanoma and the key to improved survival in all affected individuals remains early diagnosis and treatment.

One of the most important issues facing cancer biologists and medical oncologists is controlling the process of tumor metastasis. A major deterrent in studies of the metastatic process has been the sheer complexity of the process itself and frustrations in accurately modeling events leading to metastasis. Melanoma is a disease with high metastatic potential even at very early stages of development. The key to improved melanoma patient survival is early diagnosis and treatment, even in the metastatic setting. There are currently no tests to accurately predict patient outcome for early stage disease and no blood tests that readily indicate disease recurrence/progression.

Current disease monitoring is through the use of frequent physical examinations in conjunction with various imaging modalities including CT-scanning, MRI scanning, and PET scanning. Such patient monitoring techniques usually identify only grossly-detectable disease which is often difficult to treat. In addition, there is currently no simple test available to readily predict melanoma outcome or monitor disease in patients with a history of melanoma who may be at high risk for recurrent disease. In addition, there are no quantitative ways to identify patients who are at high-risk for the development of invasive melanoma.

Therefore, there still exists an unmet need to find novel targets for treating metastatic melanoma.

### SUMMARY OF THE INVENTION

The inventors of the present invention have developed a novel technology to evaluate melanoma cell interaction with endothelial cells as a marker of tumor-vessel crosstalk and metastatic potential.

In accordance with an embodiment, the present invention provides a method for detecting or diagnosing melanoma in a subject comprising: (a) obtaining a biological sample from the subject; (b) detecting the presence of i) cells expressing Notch3, ii) soluble Notch3, or iii) a soluble fragment of Notch3 in the sample; and (c) correlating the expression of Notch3 with the presence of melanoma, thereby detecting or diagnosing melanoma in a subject.

In accordance with an embodiment, the present invention provides a method of predicting recurrence of melanoma in a subject comprising: (a) obtaining a biological sample from the subject; (b) detecting the presence of i) cells expressing Notch3, ii) soluble Notch3, or iii) a soluble fragment of Notch3 in the sample; and (c) correlating the expression of Notch3 with the presence of melanoma, thereby predicting the recurrence of melanoma in a subject.

In accordance with an embodiment, the present invention provides a method of identifying a subject at risk of developing melanoma comprising: (a) obtaining a biological sample from the subject; (b) detecting the presence of i) cells expressing Notch3, ii) soluble Notch3, or iii) a soluble fragment of Notch3 in the sample; and (c) correlating the expression of Notch3 with the presence of melanoma, thereby identifying a subject at risk of developing melanoma.

In accordance with an embodiment, the present invention provides a method for screening one or more Notch3 activity inhibitor candidates comprising: 1) constructing a transformant by transfecting a host cell with a plasmid comprising a polynucleotide encoding Notch3; 2) treating the transformant with a control substance, and treating the transformant with one or more Notch3 activity inhibitor candidates (experimental group); 3) measuring Notch3 activities in the experimental group and in the control group of step 2); and 4) selecting Notch3 activity inhibitor candidates that demonstrate an inhibitory effect when compared with the control group.

### BRIEF DESCRIPTION OF THE DRAWINGS

FIG. 1 depicts the Melanoma-endothelial co-culture model schema. Metastatic melanoma cells (1205Lu) and HUVEC are fluorescently labeled to make GFP-1205Lu and RFP-HUVEC. These cells are co-cultured to 95% at 1:1 ratio in EGM-2. Each cell line is also grown alone and cultured in EGM-2, controlling for media effects on gene transcription. After 48 hrs., cells are separated by FACS which creates four populations of cells: GFP-1205Lu co-cultured, GFP-1205Lu alone, RFP-HUVEC co-cultured, RFP-HUVEC alone. The resulting populations are analyzed using global-gene microarray technology using the cells grown alone as a reference for wild-type growth.

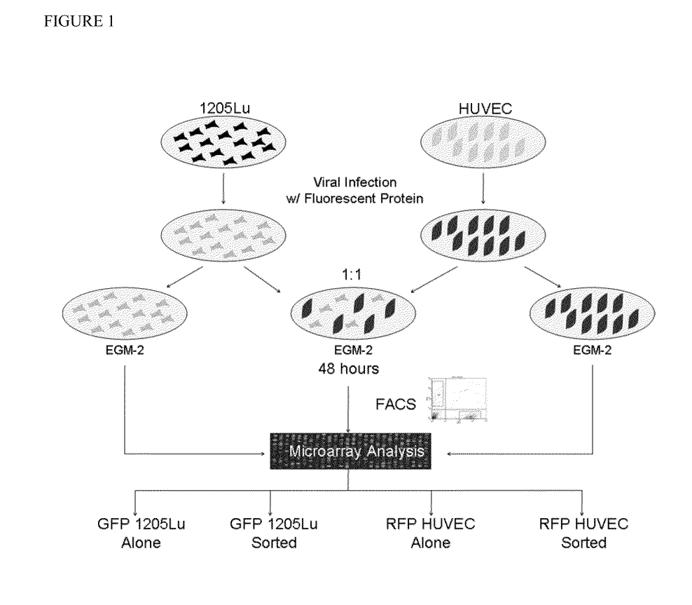

FIG. 2 shows the original microarray results regarding notch family members. Following co-culture and global gene microarray analysis, it was noted that Notch3 transcript levels were specifically upregulated in GFP-1205Lu and RFP-HUVEC sorted populations (2A). Additionally, transcript levels for a Notch ligand (JAG1, GFP-1205Lu; DLL1, RFP-HUVEC) were upregulated in each cohort, and two classical downstream targets of Notch were also upregulated in GFP-1205Lu, suggesting Notch activation (2B).

FIG. 3 shows the melanoma-endothelial co-culture experiment repeated and analyzed by qRT-PCR. To validate the microarray observations, the melanoma-endothelial co-culture experiment was repeated and gene targets were analyzed by qRT-PCR, a much more accurate means of assaying mRNA transcript levels. qRT-PCR data (filled bars) was compared to microarray data (clear bars) from the original screen.

FIG. 4 depicts qRT-PCR analysis of three phases of melanoma cell lines following RFP-HUVEC co-culture. The aforementioned melanoma cell lines were GFP-tagged and co-cultured with RFP-HUVEC for 48 hours. Following FACS, cell lines were analyzed by qRT-PCR for Notch receptor (4A) and downstream target transcript levels (4B).

FIG. 5 shows dual-luciferase assays to validate and quantify Notch signaling in the melanoma-endothelial cell co-culture. GFP-1205Lu was transiently transfected with 4×CBF-1 Notch luciferase reporter and was grown alone or with RFP-HUVEC co-culture for 24 hours (5A). DAPT, γ-secretase inhibitor, completely abrogated all Notch signaling observed, while DMSO (vehicle) had no effect. The observed signaling increased with time (5B), and could not be initiated without RFP-HUVEC (5C). A CBF-1 point mutant negative control was not activated during co-culture. *** p-value <0.001.

FIG. 6 depicts the melanoma-endothelial cell co-culture schema utilizing Notch luciferase reporters to demonstrate RFP-HUVEC dose-dependence. Using dual-luciferase reporter assays, Notch signaling was quantified within the co-culture model. To further demonstrate the requirement of RFP-HUVEC to initiate Notch signaling within the model, a dose-dependence experiment designed such that the total number of cells was conserved while changing the ratio of RFP-HUVEC to GFP-1205Lu transiently transfected with the Notch luciferase reporter.

FIG. 7 shows that notch signaling is dose-dependent with respect to RFP-HUVEC cell number and requires cell-cell contact. GFP-1205Lu was transiently transfected with the Notch luciferase reporter and co-cultured at different ratios with respect to RFP-HUVEC cell number (7A). The Notch signaling initiated was dose-dependent with respect to endothelial cell number, while a negative control CBF-1 point mutant reporter failed to quantify any Notch signaling. To determine whether any soluble factors could explain the signaling observed, two different flasks (CM1, CM2) were used to condition EGM-2 medium. These media were filtered to remove any cellular debris and incubated with GFP-1205Lu expressing the Notch luciferase reporter (7B). Notch signaling was not observed, suggesting the Notch signaling observed in the co-culture system is juxtacrine in nature. ** p-value<0.01.

FIG. 8 shows that notch inhibition disrupts RFP-HUVEC networking phenotype. GFP-1205Lu, RFP-HUVEC, and 1:1 ratio of GFP-1205Lu:RFP-HUVEC co-culture were treated with either 0.1% DMSO vehicle control (8A-8C) or 10 μM DAPT (8D-8F), effectively inhibiting all quantified Notch signaling. While no discernable difference in GFP-1205Lu or RFP-HUVEC growth was noted with DAPT treatment, merged co-culture pictures demonstrate RFP-HUVEC network disruption with respect to DMSO vehicle control.

FIG. 9 depicts Notch3 and downstream target qRT-PCR evaluation following N3ICD and shN3 expression in three phases of melanoma. GFP-Sbc12 (radial phase), GFP-WM983A (vertical phase), and GFP-1205Lu (metastatic) cell lines were stably transfected using retroviral constructs with pBABE (empty vector) and N3ICD (9A-9C). While N3ICD was accepted to varying degrees, all cell lines exhibited downstream target changes, suggesting the N3ICD exogenous protein was being expressed. The above cell lines were also stably transfected using lentiviral constructs with scramble shRNA control and shRNA Notch3 (9D-9F). All cell lines experienced efficient Notch3 transcript silencing; however the downstream target changes were not as consistent as N3ICD activation downstream profile.

FIG. 10 is a photograph of Notch3 overexpression and validation by Western blot.

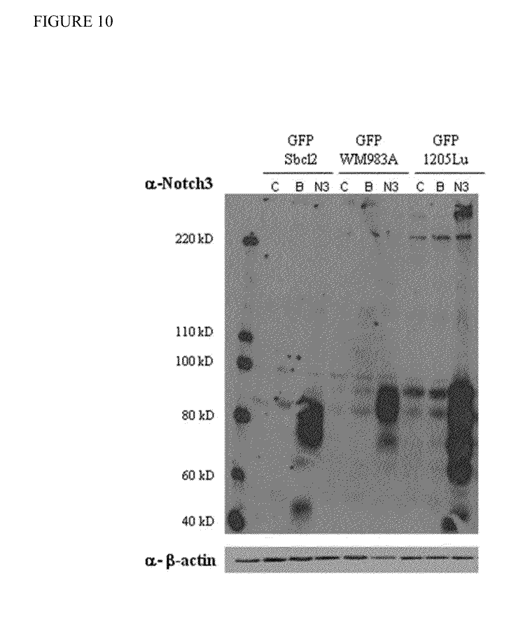

FIG. 11 shows a set of proliferation assays for Notch3 overexpression and knockdown melanoma cell lines. Following Notch3 expression validation, N3ICD (11A-11C) and shN3 (11D-11F) melanoma cells were plated in 96-well plates and allowed to proliferate for 7 days. XTT metabolism was utilized to estimate cell number. Both N3ICD and shN3 increased GFP-Sbc12 proliferation, while shN3 significantly decreased the growth rate of GFP-WM983A. * p-value<0.05, ** p-value<0.01, *** p-value<0.001.

FIG. 12 depicts representative pictures of soft agar assays for control, pBABE, and N3ICD melanoma cell lines. Control (12A-12C), pBABE (12D-12F), and N3ICD (12G-12I) expressing melanoma cells were cultured in 1% soft agar for 14-17 days and visualized with NBT. GFP-Sbc12, a radial phase melanoma, was the least clonogenic cell line, while GFP-WM983A and GFP-1205Lu, representing more advanced melanoma cell lines demonstrated a marked increase in clone formation. While N3ICD expression had little effect on GFP-Sbc12 and GFP-1205Lu clonogenicity, constitutive Notch3 activation greatly decreased GFP-WM983A clonogenic ability.

FIG. 13 depicts representative pictures of soft agar assays for control, scramble, and shN3 melanoma cell lines. Control (13A-13C), scramble (13D-13F), and shN3 (13G-13I) expressing melanoma cells were cultured in 1% soft agar for 14-17 days and visualized with NBT. Lentiviral selection greatly decreased GFP-Scb12 and GFP-1205Lu clonogenicity, however, shN3 expression slightly restored GFP-Scb12 clonal phenotype. GFP-WM983A demonstrated a dramatic, and Notch3 specific decrease in clonogenicity.

FIG. 14 shows a set of graphs of quantification of clonogenicity and colony size for melanoma cell growth in soft agar. N3ICD and shN3 expressing cells were seeded in 1% soft agar with accompanying control cell lines. Clonogenicity was defined as the average number of colonies per well (14A, B). GFP-WM983A experienced the greatest changes in clonogenicity with Notch3 status. Colony size was approximated by MCID elite and compared as box-and-whisker plots (14C, D). While, Notch3 status affected GFP-WM983A colony size in a similar manner to clonogenicity, shN3 expression decreased the average colony size in all three conditions. * p-value<0.05, *** p-value<0.001.

FIG. 15 shows transwell migration assays for Notch3 overexpression and knockdown in melanoma cell lines. Melanoma cell lines with defined Notch3 status were seeded in transwell migration assays, evaluating the ability of these cells to specifically migrate across an extracellular matrix (Matrigel) in response to a 0-10% serum gradient. N3ICD expression slightly decreased GFP-Sbc12 invasiveness, while greatly increasing the invasive capabilities of GFP-WM983A (15A). While lentiviral selection dramatically increased GFP-Sbc12 invasiveness, this gain of function was ultimately not Notch3 specific (15B). However, shN3 expression decreased GFP-WM983A invasiveness 75%. * p-value<0.05, *** p-value<0.001.

FIG. 16 depicts the Directed cell motility analysis of Notch3 effects on GFP-1205Lu migration. A newly developed technology, the directed cell motility assay provides a more accurate and biologically relevant means of quantifying cellular migration. Nanopatterned horizontal lines coated with Collagen I commit the cell to a linear path, while providing a surface common to the in vivo setting (16A). N3ICD expression increases, while shN3 knockdown decreases GFP-1205Lu migration, suggesting Notch3 is involved in regulating cellular migration plasticity (16B).

FIG. 17 shows representative pictures of benign nevus Notch3 IHC. Benign nevi were stained with a validated Notch3 antibody using standard immunohistochemical techniques. A negative control (17A) lacked any non-specific staining, while Notch3 staining (17B) was most commonly visualized in sebaceous glands.

### DETAILED DESCRIPTION OF THE INVENTION

The inventors of the present invention utilized a reductionist approach in developing a melanoma-endothelial cell co-culture system designed to elucidate molecular determinants of melanoma metastasis. One molecular mediator of melanoma-endothelial cell crosstalk uncovered by this model was Notch3, a transcript which was upregulated in both melanoma and endothelial cells following co-culture. This model was expanded beyond the scope of a single metastatic melanoma cell line, and Notch3 upregulation following endothelial cell co-culture was observed to be a common mediator of melanoma-endothelial cell crosstalk for melanoma cell lines representing the full spectrum of melanoma development. Notch signaling between metastatic melanoma and endothelial cells was quantified using dual-luciferase technology, and was shown to be dose-dependent with the number of endothelial cells. The functional consequences of Notch3 manipulation were found to be heterotypic across the three phases of melanoma, however, phenotypic differences were most consistent in the vertical phase. It was found that Notch3 expression maintains vertical phase proliferation, while mediating tumor invasiveness. Furthermore, Notch3 modulates motility of metastatic melanoma cells in a novel directed-cell migration assay. Evaluation of Notch3 expression in primary human melanocytic lesions was performed using immunhistochemistry, and elevated Notch3 expression was found to be a common feature of malignant melanomas without significant expression in benign melanocytic nevi.

In accordance with an embodiment, the present invention provides a method for detecting or diagnosing melanoma in a subject comprising: (a) obtaining a biological sample from the subject; (b) detecting the presence of i) cells expressing Notch3, ii) soluble Notch3, or iii) a soluble fragment of Notch3 in the sample; and (c) correlating the expression of Notch3 with the presence of melanoma, thereby detecting or diagnosing melanoma in a subject.

In accordance with another embodiment of the present invention, it will be understood that the term “biological sample” or “biological fluid” includes, but is not limited to, any quantity of a substance from a living or formerly living patient or mammal Such substances include, but are not limited to, blood, serum, plasma, urine, cells, organs, tissues, bone, bone marrow, lymph, lymph nodes, synovial tissue, chondrocytes, synovial macrophages, endothelial cells, and skin. Preferred samples are derived from tumor, skin and serum.

It will be understood by those of ordinary skill, that there are a number of ways to detect Notch3 expression, and these are known in the art. Examples of preferred methods of detection of Notch3 mRNA in a sample include the use of RT-PCR, qRT-PCR, and HPLC/MS methods. Other methods include assays using antibodies to Notch3, i.e., ELISA assays. Also, the antibodies to Notch3, which are commercially available can be labeled, the quantity of label detected can also be used to quantify Notch3 expression. The Notch3 expression quantification information gathered from these methods can be generated using any type of microprocessor or computing device.

In accordance with an embodiment, the antibodies which specifically bind Notch3 and are used in determining the level of expression of Notch3 can be recombinant. As used herein, “recombinant antibody” refers to a recombinant (e.g., genetically engineered) protein comprising at least one of the polypeptides of the invention and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In accordance with an embodiment, the present invention provides a method for identifying whether a subject has an increased risk of invasive metastatic melanoma comprising a) obtaining a biological sample from a subject, b) determining the level of Notch3 expression in the sample, c) comparing the level of Notch3 of the sample to the level of Notch3 expression of a control, wherein when the level of Notch3 of the sample is greater than the level of Notch3 of the control, then the subject has an increased risk of invasive metastatic melanoma.

As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein. Preferably, the cancer is a metastatic cancer.

The phrase “controls or control materials” refers to any standard or reference tissue or material that has not been identified as having cancer.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

As used herein, the term “host cell” refers to any type of cell that can contain the viral DNA disclosed herein. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, BC-3 cells, and the like. In an embodiment, the host cell is preferably a mammalian cell. Most preferably, the host cell is a human cell or human cell line. The host cell can be of any cell type, can originate from any type of tissue, and can be of any developmental stage.

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

The term “reacting” in the context of the embodiments of the present invention means placing compounds or reactants in proximity to each other, such as in solution, in order for a chemical reaction to occur between the reactants.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Included in the scope of the invention are conjugates, e.g., bioconjugates, comprising any Notch3 polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44 (15): 5405-5415 (2005)).

Further provided by the invention is a nucleic acid comprising a nucleotide sequence encoding any of the Notch3 polypeptides, or proteins described herein (including functional portions and functional variants thereof).

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL). More preferably, the host cell is a T cell.

Methods of testing antibodies for the ability to bind to any functional portion of Notch3 are known in the art and include any antibody-antigen binding assay, such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). Alternatively, other methods, such as EBV-hybridoma methods (Haskard and Archer, J. Immunol. Methods, 74 (2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

The invention also provides antigen binding portions of any of the antibodies described herein. The antigen binding portion can be any portion that has at least one antigen binding site, such as Fab, F(ab′)₂, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)). Antibody fragments of the invention, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Also provided is a method of detecting the presence metastatic melanoma in a host. The method comprises (i) contacting a sample comprising cells of the cancer any Notch3 polypeptides, proteins, nucleic acids, recombinant expression vectors, host cells, populations of cells, or antibodies, or antigen binding portions thereof, described herein, thereby forming a complex, and detecting the complex, wherein detection of the complex is indicative of the presence of melanoma in the host.

With respect to the inventive method of detecting melanoma in a host, the sample of cells of the cancer can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting step can take place in vitro or in vivo with respect to the host. Preferably, the contacting is in vitro.

Also, detection of the complex can occur through any number of ways known in the art. For instance, the Notch3 polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

It will be understood by those of ordinary skill in the art that the methods of the present invention can also be used to identify small molecule inhibitors for Notch3 expression. In accordance with an embodiment, the present invention provides a method for screening one or more Notch3 activity inhibitor candidates comprising: 1) constructing a transformant by transfecting a host cell with a plasmid comprising a polynucleotide encoding Notch3; 2) treating the transformant with a control substance, and treating the transformant with one or more Notch3 activity inhibitor candidates (experimental group); 3) measuring Notch3 activities in the experimental group and in the control group of step 2); and 4) selecting Notch3 activity inhibitor candidates that demonstrate an inhibitory effect when compared with the control group.

As used herein, the one or more candidates are selected from the group consisting of natural compounds, synthetic compounds, RNA, DNA, polypeptides, enzymes, proteins, ligands, antibodies, antigens, bacterial or fungal metabolites and biological molecules.

### EXAMPLES

Cell Culture.

Human melanoma cell lines representing radial (WM35, SBc12, WM1552C), vertical (WM1341D, WM902B, WM278, WM983A), and metastatic (WM852, WM983B, 1205Lu, WM793) phases were kindly provided by Dr. Meenhard Herlyn (Wistar Institute). All culture supplies were obtained from Gibco unless otherwise specified. Melanoma cells were cultured in DMEM supplemented with 10% fetal bovine serum (FBS; Gemini Bioproducts, West Sacramento, Calif.), 1% penicillin/streptomycin, and 1% L-glutamine. HEK293T and Phoenix cells (Ampho; Orbigen, San Diego, Calif.) were cultured in the same medium. Human umbilical vein endothelial cells (HUVEC; Cambrex, Walkersville, Md.) were cultured in EGM-2 supplemental medium (Lonza, Switzerland). All cells were incubated at 37° C. in 98% humidified air containing 5% CO₂.

For co-culture experiments, various GFP-labeled melanoma cell lines were co-cultured with RFP-HUVEC at a 1:1 ratio to 95% confluency in EGM-2. Melanoma cells grown alone were cultured in EGM-2 to control for medium effects. Cells were co-cultured for 48 hours before sorting by FACS into pure populations. The sorted cell populations were divided and stored at −80° C. for mRNA and protein analysis.

RNA Extraction and Reverse Transcription (RT).

For RNA production, cells were snap-frozen and stored at −80° C. RNA extraction was performed using the RNeasy Mini Kit (Qiagen) according to the provided protocol. cDNAs for RT-PCR and qRT-PCR analysis were synthesized using the Super Script First-Strand System (Invitrogen).

Real-Time RT-PCR Analysis of Notch Family Members.

To quantify changes in target transcript levels, cDNA (45 ng) was added to SYBR Green PCR Master Mix (Applied Biosystems) and 1:10 forward and reverse primers in a total volume of 20 μl. A 7500 Real Time PCR System (Applied Biosystems) was used, and each reaction mixture was held at 50° C. for 2 min., heated to 95° C. for 10 min., and 40 cycles of 50° C.-to-72° C. transitions were performed. Data were analyzed using 7500 System SDS Software (Applied Biosystems).

Notch Luciferase Reporter Co-Cultures.

GFP-1205Lu was transfected using Lipofectamine 2000 with 4XCBF-1 Notch luciferase reporter (kindly provided by Dr. Diane Hayward, Johns Hopkins University) and Renilla control plasmid (2 μg total cDNA/well). Cells were transfected for 4 hr, then harvested by trypsinization and co-cultured with RFP-HUVEC at varying ratios for 24 hr in EGM-2. Cells were washed with PBS, snap-frozen, and stored at −80° C. Co-cultures were lysed using the Dual-Luciferase Reporter Assay System (Promega) and analyzed on a 1450 Microbeta plate reader (Perkin Elmer). The specificity of Notch signaling was verified using the pan-Notch inhibitor DAPT and 0.1% DMSO as a vehicle control.

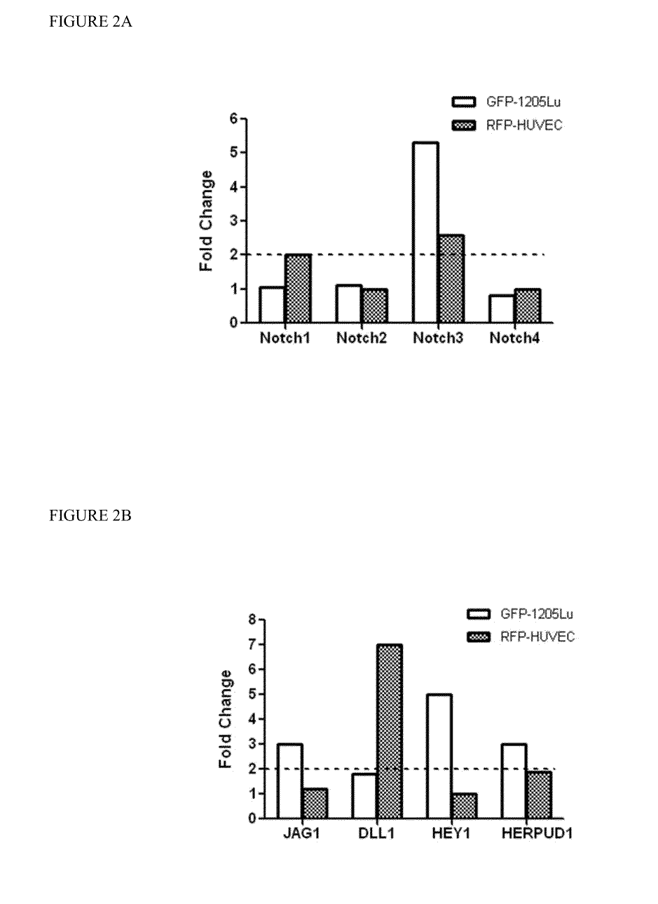

During a previous investigation, our laboratory sought to develop a simple, but clinically relevant, in vitro model system to screen for genes targets mediating melanoma metastasis. After evaluating a number of systems, the simplest model involved co-culturing fluorescently-labeled metastatic melanoma cells (GFP-1205Lu) and human umbilical vein endothelial cells (RFP-HUVEC) for 48 hr, separating them by FACS, and analyzing the sorted populations with respect to cells grown alone using global-gene microarray technology (FIG. 2.1). In doing so, we took a reductionist approach to evaluating the communication occurring at the melanoma-endothelial interface prior to a metastatic event. Initial investigations observed RFP-HUVEC patterning into highly-organized networks following co-culture with GFP-1205Lu (1). By definition, this metastatic melanoma had already shown its ability to successfully navigate the circulatory system in vivo, and it was hypothesized that the molecular memory required to communicate with endothelial cells should be retained and functional. Thus, significant changes in melanoma gene transcription following co-culture can identify novel targets for the pharmacological prevention of melanoma metastasis.

Initially, the clinical relevance of this model system was evaluated by investigating neuropilin-2 (NRP-2), a target gene upregulated in GFP-1205Lu following co-culture.

Through subsequent investigations it was demonstrated that NRP-2 is indeed positively correlated with melanoma progression and communication with endothelial cells (1). As melanocytes are derived from the neural crest (2), it was confirmed that NRP-2, an axonal-guidance protein (3), was involved in the progression of a dermatological malignancy with a shared developmental lineage. It was soon noted that Notch3 was specifically upregulated 5-fold in GFP-1205Lu and 2.5-fold in RFP-HUVEC during our initial screen (FIG. 2A, Table 1). Additionally, the Notch ligands JAG1 and DLL1 were upregulated in GFP-1205Lu and RFP-HUVEC, respectively (FIG. 2B). Notch activation initiates a cascade of downstream signaling, and these changes should be evident if the Notch pathway is truly activated in our system. Although the downstream targets of Notch signaling are cell specific (4,5), the HES and HEY transcription factors are considered classical downstream targets of Notch (6). HEY1 and HERPUD1 were also upregulated in GFP-1205Lu following co-culture (FIG. 2.2B, Table 1).

TABLE 1
The 30 most upregulated transcripts in GFP-
1205Lu following co-culture with RFP-HUVEC.

| | | Gene | |
|---|---|---|---|
| Probeset ID | Gene Title | Symbol | Fold |
| 216438_s_at | thymosin, beta 4, X-linked | TMSB4X | 36.67 |
| 205612_at | multimerin 1 | MMRN1 | 14.93 |
| 201859_at | proteoglycan 1, secretory granule | PRG1 | 14.79 |
| 214841_at | cornichon homolog 3 (Drosophila) | CNIH3 | 9.13 |
| 1556499_s_at | collagen, type I, alpha 1 | COL1A1 | 8.94 |
| 1555623_at | DERP12 (dermal papilla protein 12) | DERP12 | 7.80 |
| 232113_at | Hypothetical gene supported by | — | 7.16 |
| | BX647608 | | |
| 225566_at | neuropilin 2 | NRP2 | 6.33 |
| 823_at | chemokine (C-X3-C motif) ligand 1 | CX3CL1 | 6.31 |
| 226158_at | kelch-like 24 (Drosophila) | KLHL24 | 6.21 |
| 207147_at | distal-less homeo box 2 | DLX2 | 5.95 |
| 230538_at | rai-like protein | RaLP | 5.73 |
| 212706_at | RAS p21 protein activator 4 | RASA4 | 5.71 |
| 201667_at | gap junction protein, alpha 1, 43 kDa | GJA1 | 5.65 |
| | (connexin 43) | | |
| 201438_at | collagen, type VI, alpha 3 | COL6A3 | 5.61 |
| 237169_at | Tenascin C (hexabrachion) | TNC | 5.49 |
| 227020_at | yippee-like 2 (Drosophila) | YPEL2 | 5.47 |
| 213413_at | stoned B-like factor | SBLF | 5.46 |
| 203238_s_at | Notch homolog 3 (Drosophila) | NOTCH3 | 5.42 |
| 201858_s_at | proteoglycan 1, secretory granule | PRG1 | 5.10 |
| 209071_s_at | regulator of G-protein signalling 5 | RGS5 | 5.01 |
| 202112_at | von Willebrand factor | VWF | 4.98 |
| 238067_at | FLJ20298 protein | FLJ20298 | 4.89 |
| 225728_at | Importin 9 | IPO9 | 4.82 |
| 44783_s_at | hairy/enhancer-of-split related with | HEY1 | 4.78 |
| | YRPW motif 1 | | |
| 229225_at | neuropilin 2 | NRP2 | 4.77 |
| 226436_at | Ras association (RalGDS/AF-6) | RASSF4 | 4.75 |
| | domain family 4 | | |
| 214632_at | neuropilin 2 | NRP2 | 4.72 |
| 232797_at | Integrin, alpha V | ITGAV | 4.72 |
| 231779_at | interleukin-1 receptor-associated | IRAK2 | 4.60 |
| | kinase 2 | | |

Recombinant Lentiviruses and Retroviruses.

Various viral vectors were utilized for stable transcript expression and fluorescent cell labeling. Production of shRNA lentivirus was achieved by co-transfection of PSPAX2 and PMD2G packaging vectors with pLKO shRNA Notch3 or scrambled control vectors (Johns Hopkins HiT Center, Baltimore, Md.) using Lipofectamine 2000 (Invitrogen). Plasmids for Notch3 intracellular domain (N3ICD) or pBABE empty vector retroviral construction were provided by Dr. Ie-Ming Shih (Johns Hopkins University). Viral packaging was achieved by transfection of these plasmids into Phoenix (Ampho) cells using Lipofectamine 2000.

Viral Infection of Targeting Cells.

GFP and RFP labeling of cells was performed as previously described (18). For lentiviral infection, filtered viral medium was added to target cells cultured in serum-free Opti-MEM at various multiplicities of infection in the presence of Polybreen (8 μg/ml) to minimize off-target toxicity while maximizing target gene knockdown. Cells were selected with puromycin (1 μg/ml), and shRNA efficiency was analyzed by PCR. For retroviral infection, the same protocol was employed; however, target cells were infected three times on consecutive days. N3ICD expression was verified by qRT-PCR of known downstream targets of Notch.

Cell Proliferation Assays.

Cell proliferation was measured using sodium 3′-(1-[phenylaminocarbonyl]-3,4-tetrazolium)-bis(4-methoxy-6-nitro)benzene sulfonic acid hydrate (XTT) assay (Roche) while cells were cultured in 1% FBS DMEM. Cells plated in 96-well plates were incubated at 37° C. for 2-4 hr with 50 μl XTT reagent per 100 μl medium, and the absorbance of 100 μl of medium at 450 nm/650 nm was analyzed by spectrophotometry. Standard curves were utilized to calculate cell number over time.

Clonogenicity Assays.

The clonogenicity of various cell lines was determined by colony formation in soft agar. Agar (2% in DMEM, 2 ml/well) was added to 6-well plates. Once the agar had solidified, 2 ml of 1% agar (in DMEM containing 10,000 cells/ml) was added. Once this layer had set, 2 ml DMEM was added, and the plates were maintained under normal cell culture conditions. The medium was changed every 7 days, and cells were allowed to proliferate for 17 days, except GFP-WM983A, which was stopped at Day 14 due to overlapping colonies. Following colony formation, the medium was removed, and 0.5 ml of nitroblue tetrazolium (NBT, 1 mg/ml in PBS, Roche) was added and incubated overnight. Colony number and size were quantified using MCID Elite software (MCID, Cambridge, UK).

Matrigel Assays.

Cellular invasion was determined using a transwell migration assay (BD Biosciences, Bedford, Mass.). After reconstitution in DMEM, 25,000-30,000 cells in serum-free medium were added to the inner transwell. These wells were placed in 10% FBS DMEM for 12-24 hr, creating a growth factor gradient across the matrigel layer. After incubation, the cells were fixed with methanol, the cells that had not migrated across the membrane were removed with a cotton swab, and the remaining cells were stained with crystal violet (5 mg/ml). Transwell assays without matrigel were carried out in parallel to control for differences in inherent migratory ability. The number of cells in the test sample that crossed the matrigel layer was divided by the number that migrated in the control to calculate the percent invasiveness. Control wells were quantified using Pixcavator (Intelligent Perception, Huntington, W. Va.), whereas invasive cells were counted manually.

Directed Cell Migration Assay.

The soft polyurethane (PU) mold material consists of a functionalized precursor with an acrylate group for cross-linking, a monomeric modulator, a photoinitiator and a radiation-curable releasing agent for surface activity (19). To fabricate a sheet-type mold, the liquid mixture was drop-dispensed onto a silicon master pattern, then a flexible, transparent polyethylene terephthalate (PET) film was brought into contact with the liquid mixture. Subsequently, this platform was exposed to UV light (λ=200-400 nm) for 20 s through the transparent backplane (dose=100 mJ cm-2). After UV curing, the mold was removed from the master pattern and additionally cured overnight to terminate the remaining active acrylate groups. The resulting PU mold used in the experiment was a thin sheet with a thickness of 0.50 μm. The polymeric nanostructures with periodic ridges and grooves were fabricated onto a 22 mm×40 mm glass coverslip using UV-assisted capillary lithography. Prior to the application of the PU mold, the glass substrate was thoroughly rinsed with ethanol to remove excess organic molecules and dried in a stream of nitrogen. A small amount of the PU precursor was drop-dispensed on the substrate and a PU mold was directly placed onto the surface. The PU precursor moved into the cavity by means of capillary action and was cured by exposure to UV (λ=250-400 nm) for ˜20 s through the transparent backplane (dose=100 mJ cm-2). After curing, the mold was removed from the substrate using a sharp tweezer. The adhesion between the PU nanopatterns and the glass substrate was promoted by spin-coating a thin layer (˜200 nm) of the primer prior to dispensing of the precursor solution.

For analyzing directional migration of melanoma cell lines, an 8-well chamber slide (Lab-Tek II, 154534) was bonded to the nanopatterned coverslip using biocompatible, medical adhesive glue. Collagen type I (30 mg/ml) was coated on each well overnight and cells were seeded in each well. Cells were allowed to attach and spread overnight in a static-media environment before time-lapse capture. For live-cell imaging, the nanopatterned coverslip was placed in an environmental chamber to maintain 37° C. and 5% CO₂ while being mounted to the stage of a motorized inverted microscope (Zeiss Axiovert 200M) equipped with a Cascade 512B II CCD camera. Cellular motility was automatically imaged with SlideBook software (Intelligent Imaging Innovations, Inc.) for 5 hours at 10 minute intervals. After imaging, cells in each time-lapse image were segmented manually with the help of Matlab (The Mathworks, Natick, Mass.). Cell centroids for each single cell and trajectories were plotted with time. The mean-squared displacements of each cell centroid were obtained and averaged to calculate cell migration speed.

Immunohistochemistry.

A human melanoma TMA representing 43 individual melanomas of various subtype was kindly provided by Dr. Achim Jungbluth (Sloan Kettering Institute, New York, N.Y.). Human fallopian tube utilized as a Notch3 IHC positive control was kindly provided by Dr. Ie-Ming Shih (Johns Hopkins Institute, Baltimore, Md.). Standard immunohistochemical techniques were performed using two different rabbit polyclonal Notch3 antibodies (sc-7424, Santa Cruz Biotechnologies, Santa Cruz, Calif.; D11B8, Cell Signaling Technology, Danvers, Mass.) and the EnVision Plus detection system (Dako, Carpinteria, Calif.). Briefly, tissue sections were deparaffinized and rehydrated, followed by antigen retrieval in citrate buffer. Primary Notch3 antibody was applied overnight followed by application of anti-rabbit HRP conjugated secondary antibody for 30 minutes. AEC chromogen was used rather than DAB to distinguish positive Notch3 staining from melanin in the tissues. A positive score was defined as a tumor that exhibited Notch3 staining with both antibodies.

### Example 1

Validation of Initial Microarray Observations.

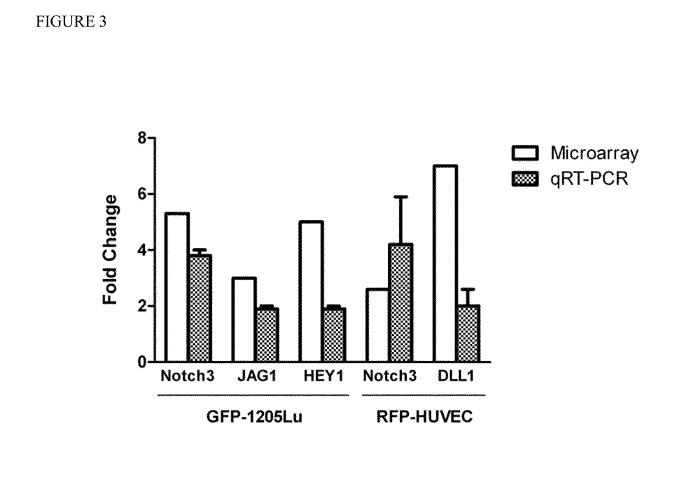

To validate Notch3 as a target gene within our melanoma-endothelial co-culture model, the initial microarray experiment was repeated. After being co-cultured for 48 hr. then sorted by FACS, GFP-1205Lu and RFP-HUVEC transcript levels were analyzed by qRT-PCR for more accurate quantification of target gene modulation. Co-cultured GFP-1205Lu experienced a 4-fold increase in Notch3 transcript with respect to cells grown alone (FIG. 3). In addition, JAG1 exhibited a 2-fold increase in transcript levels. While Notch3 and JAG1 upregulation agreed nicely with the previous microarray findings, HEY1 failed to match the extent of prior modulation. Despite the decrease in magnitude, HEY1 still exhibited a 2-fold increase in transcript levels, the previously defined threshold for genes of interest. Co-cultured RFP-HUVEC transcript levels also differed slightly from the prior microarray observations. While Notch3 levels were comparable to the initial findings, DLL1 modulated but not to the degree previously observed (2-fold vs. 7-fold change).

### Example 2

Notch3 Transcript is Upregulated in Various Phases of Melanoma Following Melanoma-Endothelial Cell Communication.

To determine whether Notch3 mRNA upregulation is a general feature of melanoma-endothelial crosstalk, rather than an artifact of a single cell line, three radial phase (WM35, SBc12, WM1552C), five vertical phase (WM1341D, WM902B, WM278, WM983A), and two additional metastatic melanoma cell lines (WM852, WM983B) were GFP-tagged and co-cultured with RFP-HUVEC (Table 2). After 48 hrs., the cells were separated by FACS as before, and protein and RNA were made for the four resulting groups of cells for each cell line: GFP-alone, GFP-sorted, with matched RFP-alone and RFP-sorted samples. Having established these important reagents, future qRT-PCR and Western blot investigations of other target genes were able to be initiated with a broad panel of co-cultured melanoma cells, rather than a specific metastatic cell line.

TABLE 2
Growth phase characteristics of melanoma cell
lines used in co-culture experiments.

| Cell Line | Phase | Pathology |
|---|---|---|
| WM35 | Radial-like | RGP, SSM, stage1 |
| SBcl2 | Radial-like | Primary |
| WM1552C | Radial-like | RGP/VGP, SSM, stage 3 |
| WM1341D | Early, vertical-like | RGP/VGP, LMM, stage n/a |
| WM902B | Late, vertical-like | RGP/VGP, LMM, stage n/a |
| WM278 | Late, vertical-like | VGP, NM, stage 2 |
| WM983A | Late, vertical-like | URGP/VGP, stage n/a |
| WM793 | Late, vertical-like | |
| WM852 | Metastatic | VGP, NM, stage 3 |
| WM983B | Metastatic | Lymph node (met. of Mel 7) |
| 1205 Lu | Metastatic | Lung (Mel 11 selected in |
| | | Mouse) |

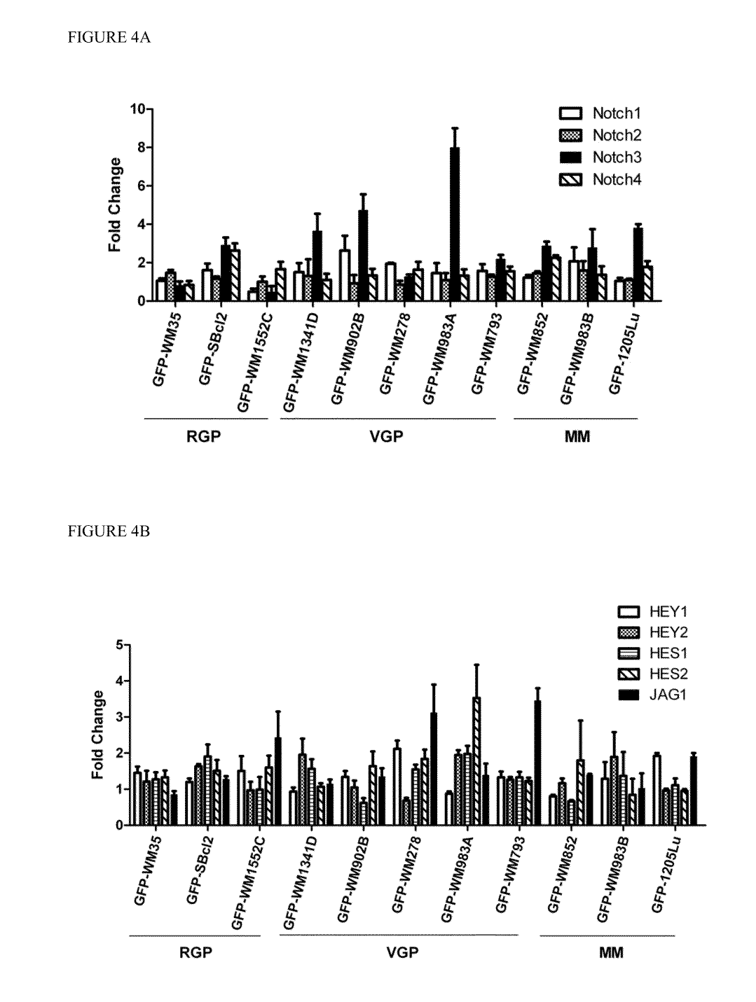

Following co-culture of the melanoma panel, transcript levels for all four Notch receptors were analyzed by qRT-PCR and Notch3 was the only receptor that consistently increased mRNA expression (FIG. 4A). While each metastatic cell line upregulated Notch3 transcript to some degree (3/3 cell lines), the vertical phase cell lines exhibited the greatest modulation in Notch3 transcript levels. Radial growth cells showed little change in Notch3 transcript following co-culture (1/3 cell lines), suggesting a phase specific response to Notch3 transcript modulation. Additionally, it was hoped our screen would highlight genes responsible for the transition from vertical to metastatic growth within the perivascular niche. Such an observation could indicate Notch3 does indeed fill this role.

Changes in Notch downstream targets and family members were also analyzed by qRT-PCR (FIG. 4B). Once again, the vertical phase cell lines showed the greatest modulation in transcript levels, however the magnitude of these changes were modest at best. Although JAG1 does not represent a classical downstream target of Notch, it was the most consistently upregulated transcript. One explanation for the lack of downstream signaling observed across the panel of melanoma co-cultured cells involves the local vs. global paradigms of cell signaling.

### Example 3

Quantification of Melanoma-Endothelial Cell Notch Signaling.

The transcriptional activity of Notch is mediated by the CBF-1 promoter sequence upstream of target genes. CBF-1 recruits CSL transcriptional repressors which inactivate gene expression in the absence of NICD. However, when NICD binds the CSL proteins, this complex transforms from a transcriptional repressor to an activator, initiating target gene synthesis. Thus, by utilizing a four-tandem repeat of CBF-1 to drive luciferase expression during a transient transfection, canonical Notch signaling can be directly quantified by chemiluminescence activity in a target cell population. FACS is not required to analyze the target cells, as any luciferase present in the lysate originated with the transfected cells of interest.

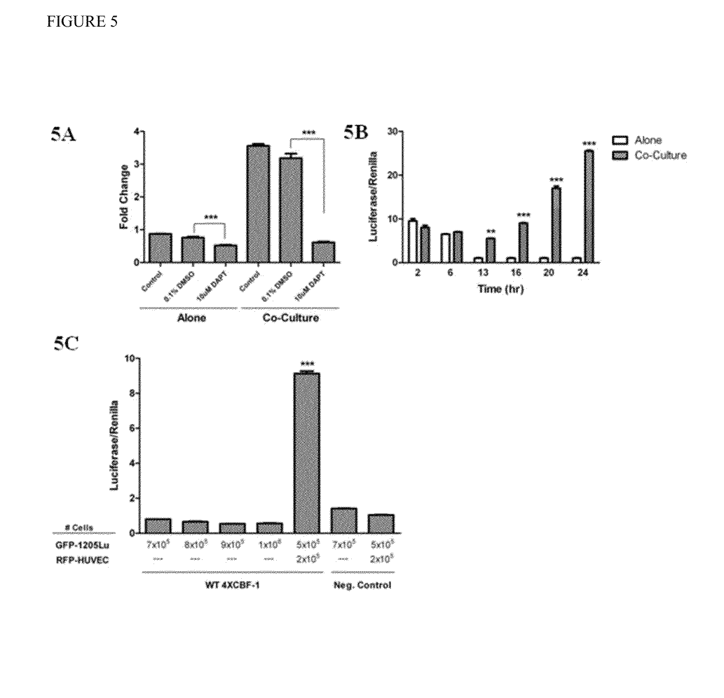

To quantify Notch signaling within the model system, GFP-1205Lu was transiently transfected with 4XCBF-1 Notch luciferase reporter (PJH23A) and co-cultured with RFP-HUVEC for 24 hours. GFP-1205Lu Notch signaling increased 4-fold in response RFP-HUVEC co-culture (FIG. 5A). DAPT, a γ-secretase inhibitor capable of inactivating all four isoforms of Notch, completely abrogated the co-culture stimulated Notch signaling. This activation also increased with time (FIG. 5B), however, the ratio of luciferase/renilla begins to decrease after 24 hours. Ultimately, the rate of luciferase production is outpaced by the Renilla transfection control due to its strong, constitutively active promoter (data not shown).

Notch signaling was virtually absent in reporter transfected melanoma cells grown alone, thus RFP-HUVEC must be the ligand-presenting cell initiating Notch activation. However, if GFP-1205Lu were sufficiently confluent, it was that Notch signaling could be stimulated without endothelial cells present. GFP-1205Lu was transiently transfected with the Notch luciferase reporter and cells were plated as dense as 1×10⁶ cells/6-well plate. PJH25A, an inactive CBF-1 point mutant, was employed as a negative control. Notch signaling was not observed in melanoma cells grown alone regardless of cell density (FIG. 5C).

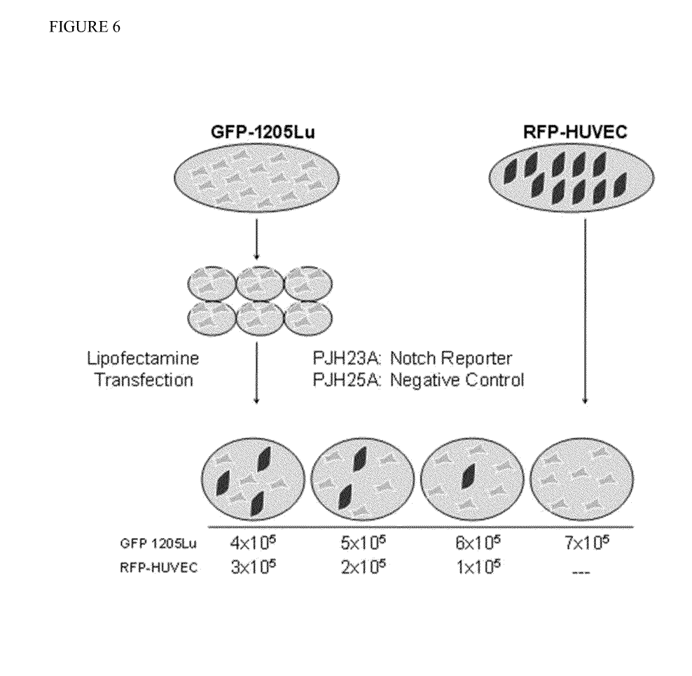

Consequently, GFP-1205Lu Notch signaling within our co-culture model is clearly endothelial-cell dependent. Therefore, the amount of Notch signaling quantified should be dose-dependent with the number of RFP-HUVEC present in the experiment. Co-cultures of the same total number of cells were established in which the ratio of RFP-HUVEC to reporter transfected GFP-1205Lu was increased in a step-wise fashion (FIG. 6).

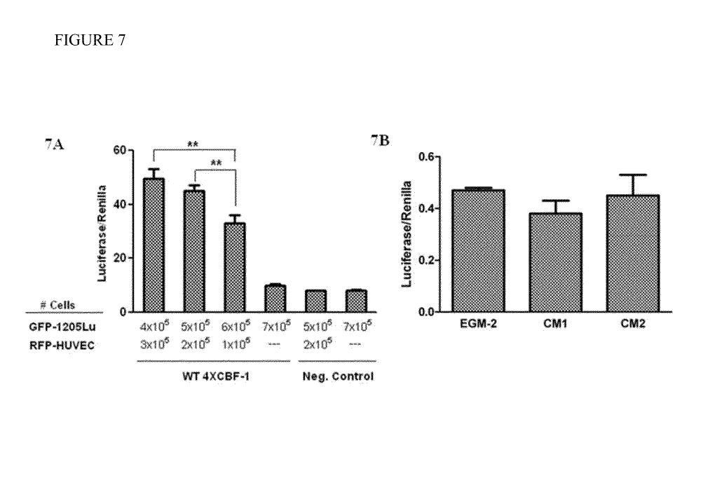

As outlined above, Notch activation involves juxtacrine signaling, where a ligand-presenting cell makes physical contact with a ligand-receiving cell. To demonstrate the Notch signaling observed is not the result of an additional, soluble factor, EGM-2 media was conditioned with RFP-HUVEC for 48 hrs. The conditioned media was recovered then syringe filtered to remove any cellular debris while preserving the proteins in solution. Subsequently, GFP-1205Lu was transfected with the Notch luciferase reporter and cultured in RFP-HUVEC conditioned media for 24 hrs. Two different flasks of RFP-HUVEC conditioned media failed to elicit any Notch signaling in GFP-1205Lu (FIG. 7).

### Example 4

Notch Signaling is Required for the RFP-HUVEC Networking Phenotype.

In previous investigations, we observed RFP-HUVEC communicating in a concerted fashion with metastatic melanoma cells to create a networking phenotype during 48 hrs of co-culture. It was thought that understanding the molecular determinants of this communication could lead to pharmacological targets for the prevention of melanoma-endothelial crosstalk in the clinical setting. To determine whether Notch signaling is involved in mediating the networking phenotype, GFP-1205Lu and RFP-HUVEC were co-cultured in 0.1% DMSO (vehicle) or 10 μM DAPT. These cells were also cultured alone in identical media to determine whether these conditions elicited any aberrant growth not attributable to co-culture.

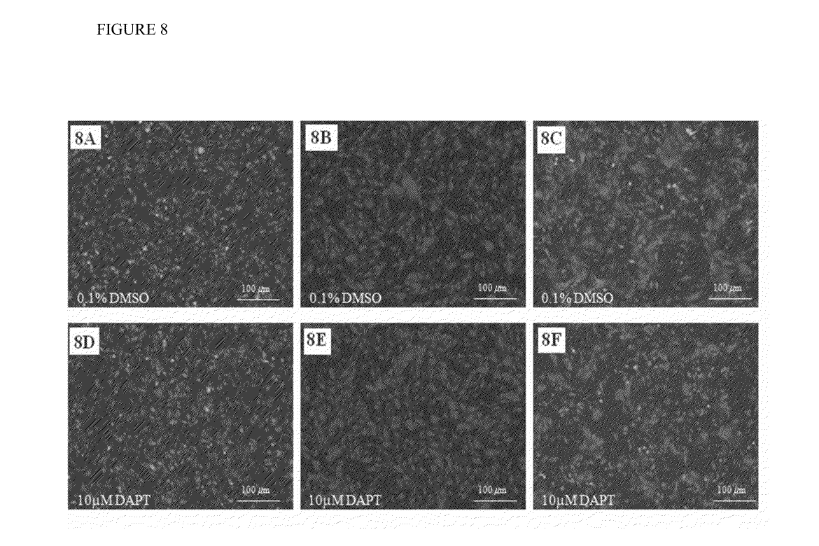

While DAPT had little effect on cells grown alone, the networking phenotype associated RFP-HUVEC-melanoma co-culture was drastically affected by Notch inhibition (FIG. 8). Rather than organizing into networked strands, Notch inhibition led to proliferative islands of RFP-HUVEC within the melanoma co-culture. Though DAPT treatment does not elucidate whether one or both cell populations require Notch signaling to achieve the networking phenotype, it does suggest the pathway is involved in mediating this communication.

### Example 5

Validation of N3ICD Overexpression and Notch3 Knockdown Cell Lines.

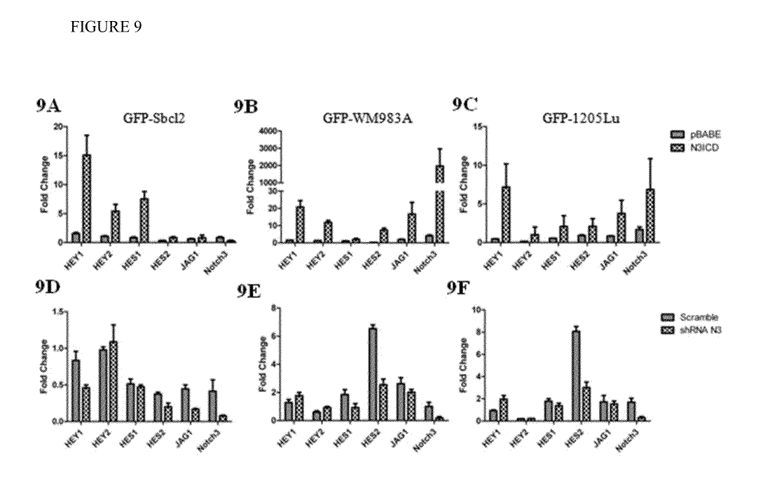

To functionally evaluate the role of Notch3 in melanoma, a cell line from each growth phase (early: GFP-Sbc12, advanced: GFP-WM983A, and metastatic: GFP-1205Lu) was chosen from the melanoma panel for stable Notch3 overexpression and knockdown. Retroviral vectors were utilized to stably introduce the active intracellular portion of Notch3 (N3ICD), previously shown to recapitulate Notch3 receptor activation. Additionally, lentiviral technology was employed to express Notch3 shRNA constructs to stably knockdown Notch3 expression (shN3) in the same cell lines. Notch3 overexpression and knockdown with respect to empty vector infection controls was confirmed by qRT-PCR and Western blot (FIG. 9, 10).

GFP-Sbc12 N3ICD exhibited a decrease in Notch3 mRNA expression, despite demonstrating a marked increase in protein concentration. It should be noted that following viral infection, the total Notch3 mRNA signal is a combination of endogenous and exogenous Notch3 transcript.

### Example 6

Evaluating Downstream Targets of Targeted Notch3 Alteration in Melanoma Cell Lines.

Target genes of Notch modulation are highly variable and cell-type dependent. However, when evaluating gene changes following Notch manipulation, isoforms of the classical Notch downstream targets HES and HEY are initially assessed. Consequently, the transcriptional status of HES and HEY family members were analyzed by qRT-PCR following stable N3ICD expression in the previously described melanoma cell lines (FIG. 9A-C). JAG1 transcript levels were also assessed, as this Notch family gene was upregulated in GFP-1205Lu following RFP-HUVEC co-culture.

HEY1 represented the most consistent downstream target of Notch3 activation, exhibiting impressive increases in transcript levels across all three phases of melanoma growth. The remaining transcript changes proved to be more phase specific, as HEY2 and HES1 activation was observed in the early and advanced cell lines, whereas HES2 and JAG1 activation was detected in the advanced and metastatic cell lines. It is interesting to note that changes in the downstream transcriptional profile of GFP-1205Lu N3ICD matched the GFP-1205Lu co-culture signaling profile quite well.

The evaluation of downstream transcriptional targets is not as straightforward following a stable decrease of Notch3 expression in the same melanoma cell lines. Although control lentiviral infection induces a substantial increase of HES2 transcript levels in two of the three cell lines (scrambled vs. control), a comparably drastic decrease is noted upon introduction of the Notch3 shRNA constructs in all three phases (FIG. 9D-F). Additionally, while N3ICD expression increased HEY1 transcript levels across all three melanoma cell lines, decreasing Notch3 expression only decreased HEY1 mRNA expression in GFP-Sbc12. In fact, HEY1 transcript levels doubled in GFP-1205Lu shN3. It is also interesting that while N3ICD expression modulated JAG1 transcript levels positively in advanced and metastatic melanoma cell lines, Notch3 knockdown decreased JAG1 mRNA in the radial phase (GFP-Sbc12) only. Thus, not only are downstream effects of Notch modulation specific to respective cancers, but they are also specific to particular phases within the disease.

### Example 7

Targeted Alteration of Notch3 Expression in Melanoma Cell Lines Results in Phase-Specific Responses in Melanoma Cell Proliferation.

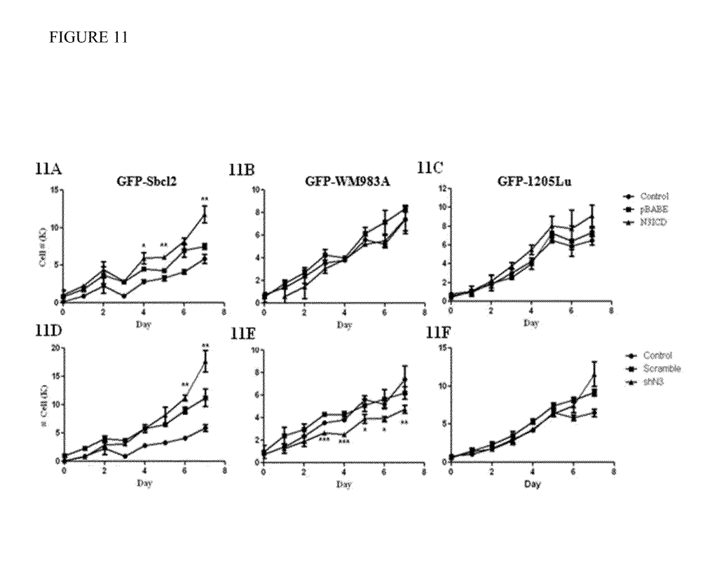

Following stable overexpression and knockdown validation, the functional aspects of Notch3 in early, advanced, and metastatic melanoma cells were evaluated. To determine whether Notch3 affects the proliferation of melanoma cell lines, growth curves were seeded in 96-well plates and proliferation was estimated by XTT metabolism with respect to a standard curve (FIG. 11).

Notch3 knockdown dramatically decreased the growth of GFP-WM983A very early, and ultimately inhibited growth by almost 50% by Day 7 (FIG. 11E). While Notch3 manipulation in radial phase melanoma exhibits conflicting results, Notch3 expression plays a role in maintaining, but not enhancing, growth during the vertical phase of melanoma development.

### Example 8

Notch3 Plays a Heterotypic Role in Vertical Phase Melanoma Clonogenicity and Clone Size.

Due to the heterogeneous nature of many tumors, an important aspect of a burgeoning tumor is the probability any one cell within the mass is capable of creating a distant metastasis within a new microenvironment in an anchorage-independent manner. Termed clonogenicity, this characteristic often increases with tumor progression, as more individual cells within the mass acquire the mutations making metastasis more likely. To evaluate the role of Notch3 in melanoma clonogenicity, N3ICD and shN3 melanoma cells were seeded in 6-well plates at a very low density (1×10⁴ cells/ml) in 1% soft agar and allowed to proliferate 14-17 days. Cells were stained and growth arrested with nitro blue tetrazolium (NBT) and scanned for colony frequency and size.

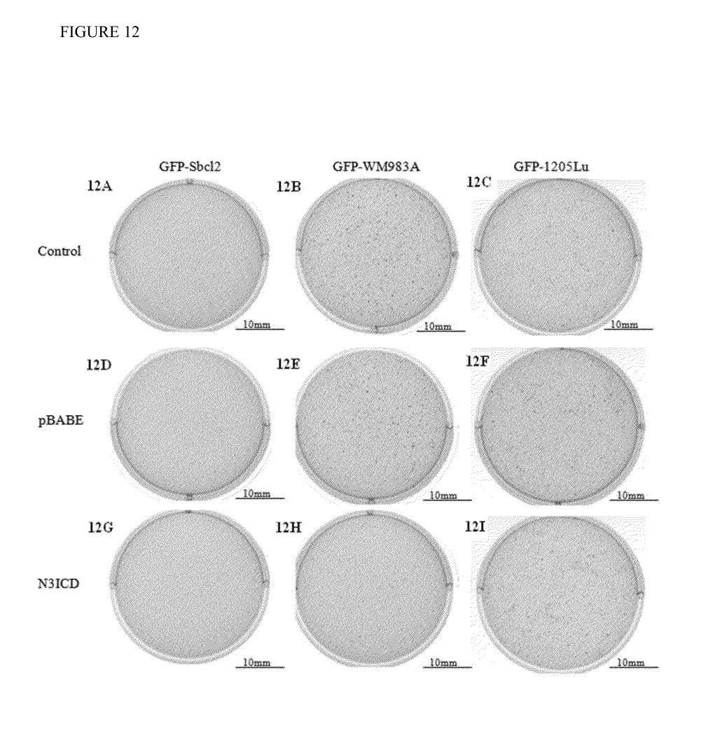

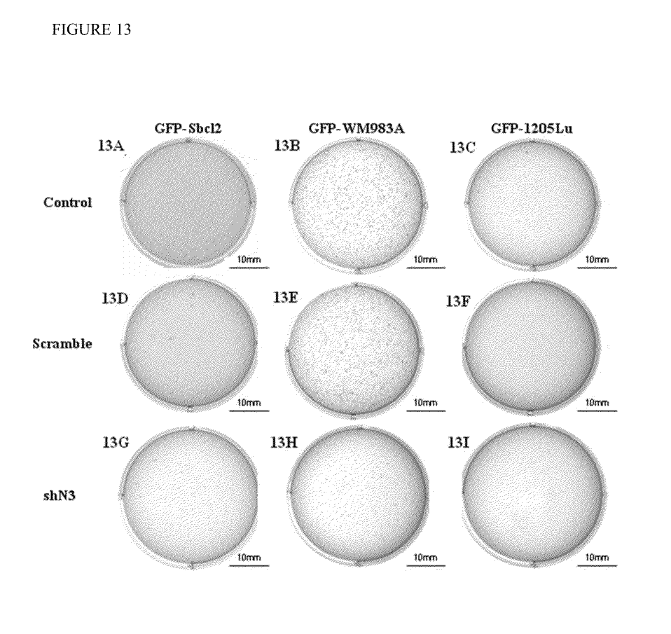

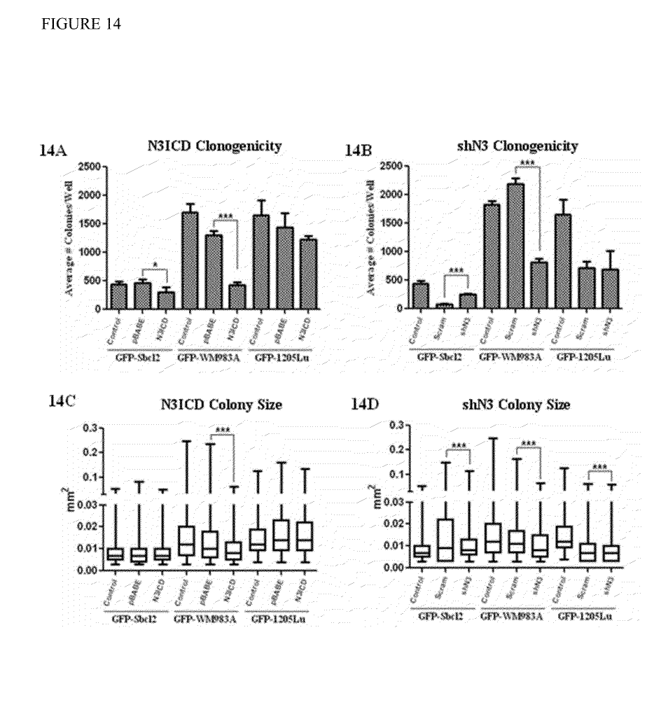

As expected, GFP-Sbc12, representing an early, radial growth phase melanoma, was the least clonogenic cell line, while GFP-WM983A and GFP-1205Lu were able to colonize the soft agar more easily (FIG. 12). Accordingly, these vertical and metastatic melanomas also created much larger colonies on average than GFP-Sbc12 (FIG. 14). However, N3ICD expression and Notch3 knockdown both decreased GFP-WM983A clonogenicity and colony size (FIGS. 12-14). N3ICD expression appeared to have similar effects on GFP-Sbc12 and GFP-1205Lu clonogenicity, however, these differences were not statistically significant. Indeed, Notch3 knockdown actually increased GFP-Sbc12 clonogenicity slightly.

The most consistent result involves the role of Notch3 knockdown on melanoma soft agar colony size. While Notch3 manipulation in GFP-WM983A recapitulated the biphasic pattern observed in clonogenicity, shN3 expression decreased average colony size in every phase of melanoma growth. Although GFP-Sbc12 colony number increased with shN3 expression, the size of these colonies was unimpressive. Additionally, while GFP-1205Lu shN3 did not experience a statistically meaningful change in clonogenicity, the average size of these colonies did decrease slightly.

### Example 9

Notch3 Mediates Invasiveness in Vertical Phase Melanoma.

A classical hallmark of cancer is the ability to alter cell adhesion properties, remodel the site of primary tumor growth, and escape to establish new colonies elsewhere within the body. Although Notch signaling does not directly play a physical or enzymatic role in any of these processes, downstream targets of Notch affecting these phenotypes have been identified.

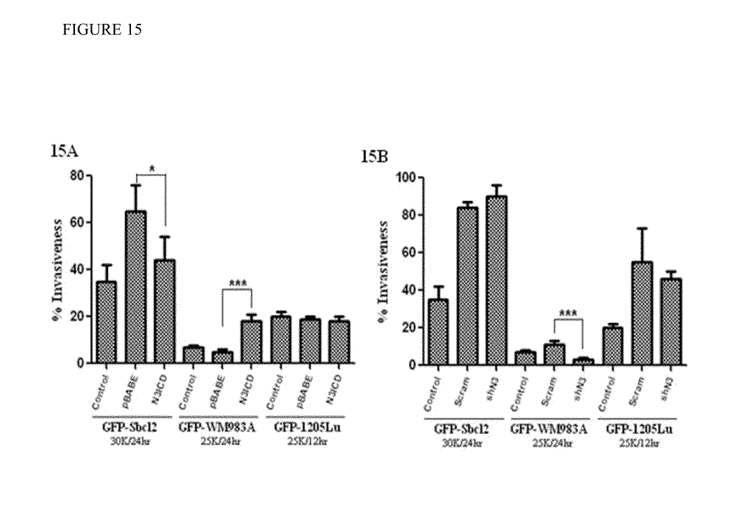

To determine whether Notch3 plays a role in mediating melanoma invasion, N3ICD and shN3 melanoma cells were seeded in transwell migration assays, challenging the cells to migrate across an extracellular protein matrix through a membrane interspersed with 10 μm pores. Employing a 0-10% serum gradient to stimulate migration, these cells were compared to identical cohorts migrating across the transwell membrane without the protein matrix to define the percentage of invasive cells. As representatives of different melanoma phases, each cell line possesses a different invasive capability. As such, each cell line required slightly different starting concentrations and incubation times to collect a significant number of invasive cells. For example, the metastatic cell line GFP-1205Lu was appropriately the most invasive, exhibiting 20% invasiveness after 12 hr beginning with 25,000 cells; whereas GFP-WM983A resulted in 7% invasiveness after 24 hr starting with an equivalent number of cells (FIG. 15). Radial phase GFP-Sbc12 demonstrated 35% invasiveness, however, this required 30,000 cells and 24 hr incubation.

With respect to Notch3 status, GFP-WM983A showed the most dramatic changes, with N3ICD causing a 250% increase in invasion, while Notch3 knockdown decreased invasiveness 75% (FIG. 15). Interestingly, GFP-Sbc12 invasion increased considerably following viral infection, however, this difference was not Notch3 specific. In fact, GFP-Sbc12 N3ICD showed a decrease in invasive potential with respect to empty vector control. Considering GFP-1205Lu showed no significant changes in invasion regardless of Notch status, the role of Notch3 in mediating melanoma invasiveness is multi-factored and highly phase specific.

### Example 10

Notch3 Mediates Motility in Metastatic Melanoma During Directed-Cell Migration.

The ability of tumor cells to remodel their microenvironment and colonize distant sites relies on their inherent motility. Thus, any genetic alterations that lead to an increase in tumor cell motility signifies and advancement in tumor progression. A common means of assessing cancer cell motility involves the “scratch assay”, in which cancer cells are grown to confluency and a vertical line is physically scratched through the lawn of cells. The time required for these cells to heal this “wound” in a low-serum environment is a direct measure of the average cell motility.

However, this assay is not ideal for a number of reasons. First, cellular migration across plastic does not adequately recapitulate the mechanical stresses experienced in the tumor microenvironment. Additionally, physical disruption of the culture destroys cells, releasing their intracellular contents into the medium, and tearing adhesive junctions maintained at the border. The directed-cell motility assay involves seeding cells on a nanopatterned coverslip of parallel lines coated with Collagen I, an extracellular matrix protein (FIG. 3.8A). The benefits of this assay are two-fold: 1.) Rather than approximate the probabilistic wanderings of cells, the directed-cell motility assay simplifies cell velocity calculation by exploiting an aligning behavior exhibited by cancer cells termed contact guidance. Mediated by integrins and Rho/ROCK cytoskeletal orientation, contact guidance allows for a directional persistence in cell invasion. 2.) Collagen I provides a surface better mimicking the biophysical properties a cell is likely encounter in the tumor microenvironment.

For example, in previous investigations radial growth phase (WM35) and metastatic melanoma (1205Lu) cell lines had comparable velocities when migrating across a standard, in vitro plastic surface. However, when seeded on the directed-cell migration matrix, the metastatic melanoma cell line traveled much faster than its radial counterpart, reestablishing their phenotypic differences in vitro (data not shown).

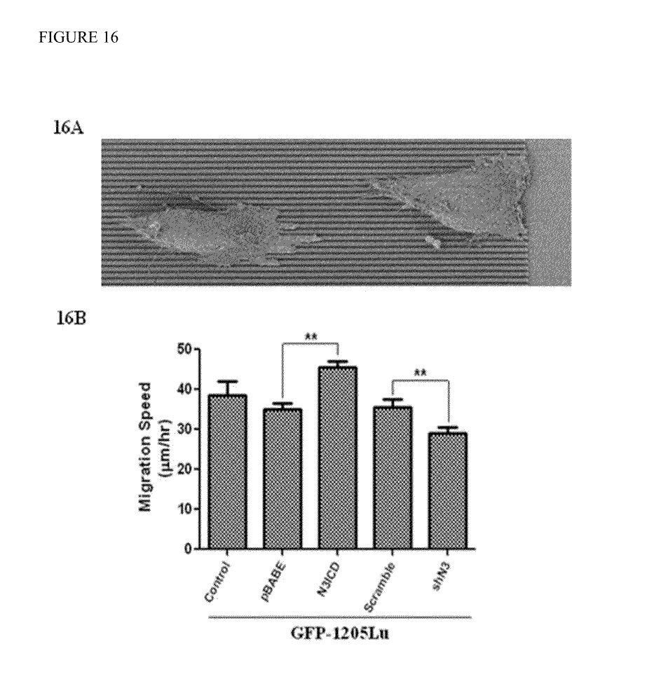

To determine whether Notch3 affects metastatic melanoma migration, N3ICD and shN3 expressing GFP-1205Lu were seeded on the directed-cell migration matrix and time-lapse photography was used to calculate the average cell velocity. N3ICD expression increased GFP-1205Lu velocity 29%, while Notch3 knockdown decreased velocity 15% (FIG. 16). Consequently, Notch3 activation confers a more advanced migratory phenotype to GFP-1205Lu, while concomitant decrease of Notch3 expression appears to retard metastatic melanoma motility. Additional experiments evaluating the role of Notch3 in mediating GFP-Sbc12 and GFP-WM983A motility are forthcoming. These findings will elucidate the broader role of Notch3 in melanoma motility, and provide the first investigations of a molecular determinant of cellular motility within the context of the directed-cell migration assay.

### Example 11

Immunohistochemical Analysis of Notch3 in Human Metastatic Melanoma and Benign Nevi.

To characterize the Notch3 antibody for TMA staining (sc-7424, Santa Cruz), normal fallopian tube and benign nevi were probed using standard IHC techniques. The epithelial lining of normal fallopian tube serves as a positive control for Notch3 expression (personal communication: Dr. Ie-Ming Shih), and accordingly this staining pattern was achieved (data not shown).

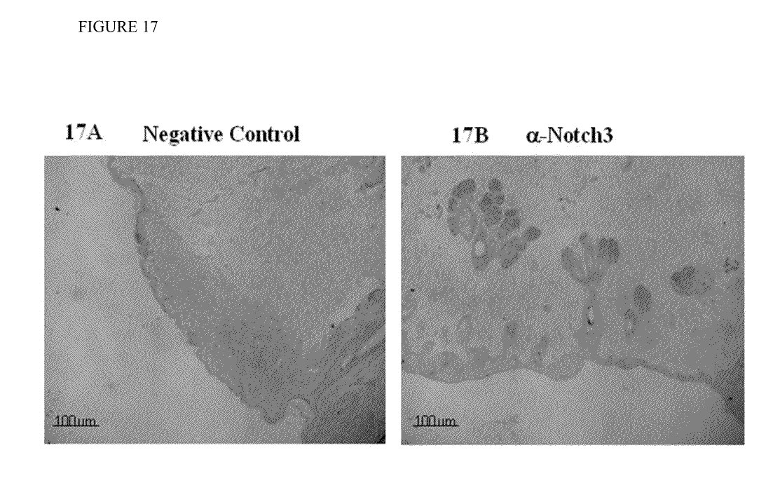

This antibody was then utilized to determine whether benign nevi are positive for Notch3 expression. While benign nevi were negative for Notch3 expression, sebaceous glands were highly positive (FIG. 17). To our knowledge, no immunohistochemical investigations of Notch receptor expression have been completed. However, Msx2-Cre γ-secretase mosaic loss-of-function mice fail to form sebaceous glands within the skin. This does not establish sebaceous glands as an internal control for Notch3 expression, but only serves to highlight the known importance of Notch signaling within this organ.

Once the antibody had been characterized, a melanoma TMA was probed utilizing the same immunohistochemical techniques. An additional Notch3 antibody (D11B8, Cell Signaling Technology) was also utilized for analytical comparison, which showed greater Notch3 specificity but lower intensity signal by Western blot. All four pathological designations present in the TMA stained positive for Notch3 to varying degrees (FIG. 17; Table 3). While 38% of melanotic malignant melanomas (8/21) stained positive for Notch3 expression, 60% of metastatic melanotic melanomas (3/5) exhibited elevated levels of Notch3 protein. Additionally, amelanotic metastatic epithelioid melanomas were 78% positive (7/9) for Notch3 expression, and these tumors demonstrated the most intense staining of any sub-group within the TMA. Interestingly, spindle-cell tumors of the same subtype were only 25% positive (2/8).

TABLE 3
Results of Notch3 IHC tissue microarray staining.

| Pathology | Positive/Total Samples | % Positive |
|---|---|---|
| Melanotic Malignant Melanoma | 8/21 | 38% |
| Metastatic Malignant | 3/5 | 60% |
| Metastatic Amelanotic | 2/8 | 25% |
| Spindle Cell Melanoma | | |
| Metastatic Amelanotic | 7/9 | 78% |
| Epithelioid Melanoma | | |

To determine the extent to which Notch3 expression does play a role in vivo, a melanoma TMA was probed using IHC to visualize Notch3 protein levels. Interestingly, while malignant melanomas stained positive to varying degrees, benign nevi did not. Metastatic melanomas were more positive than malignant melanomas (60% vs. 38%), however this could be a function of the lower number of melanotic malignant melanomas. When metastatic melanomas are grouped regardless of melanin-status, Notch3 positivity is still higher in this more advanced group (55%) than the malignant melanomas.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for detecting or diagnosing melanoma in a subject comprising:
(a) obtaining a biological sample from the subject;
(b) detecting the presence of i) cells expressing Notch3, ii) soluble Notch3, or iii) a soluble fragment of Notch3 in the sample; and
(c) correlating the expression of Notch3 with the presence of melanoma, thereby detecting or diagnosing melanoma in a subject.

2. The method of claim 1, wherein the biological sample is taken from the skin, blood, serum or tumor.

3. The method of claim 2, further comprising comparing the level of expression of Notch3 in the subject to the level of expression in one or more control samples, and when the level of expression of Notch3 is higher in the subject than the control levels, an indication of the presence of melanoma is made.

4. The method of claim 3, wherein the detection of Notch3 expression is made by the use of monoclonal antibodies to Notch3 having a detectable label.

5. The method of claim 3, wherein the detection of Notch3 expression is made by the use of an ELISA assay.

6. A method of predicting recurrence of melanoma in a subject comprising:
(a) obtaining a biological sample from the subject;
(b) detecting the presence of i) cells expressing Notch3, ii) soluble Notch3, or iii) a soluble fragment of Notch3 in the sample; and
(c) correlating the expression of Notch3 with the presence of melanoma, thereby predicting the recurrence of melanoma in a subject.

7. The method of claim 6, wherein the biological sample is taken from the skin, blood, serum or tumor.

8. The method of claim 7, further comprising comparing the level of expression of Notch3 in the subject to the level of expression in one or more control samples, and when the level of expression of Notch3 is higher in the subject than the control levels, an indication of the presence of melanoma is made.

9. The method of claim 8, wherein the detection of Notch3 expression is made by the use of monoclonal antibodies to Notch3 having a detectable label.

10. The method of claim 8, wherein the detection of Notch3 expression is made by the use of an ELISA assay.

11. A method of identifying a subject at risk of developing melanoma comprising:
(a) obtaining a biological sample from the subject;
(b) detecting the presence of i) cells expressing Notch3, ii) soluble Notch3, or iii) a soluble fragment of Notch3 in the sample; and
(c) correlating the expression of Notch3 with the presence of melanoma, thereby identifying a subject at risk of developing melanoma.

12. The method of claim 11, wherein the biological sample is taken from the skin, blood, serum or tumor.

13. The method of claim 12, further comprising comparing the level of expression of Notch3 in the subject to the level of expression in one or more control samples, and when the level of expression of Notch3 is higher in the subject than the control levels, an indication of the presence of melanoma is made.

14. The method of claim 13, wherein the detection of Notch3 expression is made by the use of monoclonal antibodies to Notch3 having a detectable label.

15. The method of claim 13, wherein the detection of Notch3 expression is made by the use of an ELISA assay.

16. A method for screening one or more Notch3 activity inhibitor candidates comprising:
1) constructing a transformant by transfecting a host cell with a plasmid comprising a polynucleotide encoding Notch3;
2) treating the transformant with a control substance, and treating the transformant with one or more Notch3 activity inhibitor candidates (experimental group);
3) measuring Notch3 activities in the experimental group and in the control group of step 2); and
4) selecting Notch3 activity inhibitor candidates that demonstrate an inhibitory effect when compared with the control group.

17. The method for screening one or more Notch3 activity inhibitor candidates according to claim 16, wherein the one or more candidates are selected from the group consisting of natural compounds, synthetic compounds, RNA, DNA, polypeptides, enzymes, proteins, ligands, antibodies, antigens, bacterial or fungal metabolites and biological molecules.
